(19) United States
(12) Patent Application Publication
(10) Pub. No.: US 2004/0019121 A1
(43) Pub. Date: Jan. 29, 2004

(54) Protein prenyl transferase inhibitors in the treatment of neuroinflammatory disease

(76) Inventors: Peter Adamson, Oxfordshire (GB); John Greenwood, Camberwell (GB)

Correspondence Address: Joseph T Leone, DeWitt Ross & Stevens, Intellectual Property Department, 8000 Excelsior Drive Suite 401, Madison, WI, 53717-1914, US

(21) Appl. No.: 10/381,492

(22) PCT Filed: Sep. 26, 2001
(86) PCT No.: PCT/GB01/04291

(30) Foreign Application Priority Data
Sep. 29, 2000 (GB) 0023915.2

(51) Int. Cl. A61K 31/045

(52) U.S. Cl. 514/739

(57) ABSTRACT

A protein prenyl transferase inhibitor is used in the treatment of neuroinflammatory disease such as MS, uveitis, alzheimers or neuroAIDS. The inhibitor may be a combination of a farnesyl transferase inhibitor and a geranylgeranyl transferase inhibitor. Preferably, FTI-277 and/or GGTI-298 is employed.

This invention relates to the treatment of neuroinflammatory disease. Neuroinflammation is taken to mean aberrant trafficking of leukocytes across blood-tissue barriers of the central nervous system (CNS).

We have found that neuroinflammatory disease may be alleviated by the administration of one or more inhibitors of protein prenylation. As explained more fully hereinafter, these inhibitors prevent the post-translational prenylation of endothelial Rho proteins which is normally necessary for the functional activity of such proteins as signalling molecules in promoting the endothelial migration of T-lymphocytes and leukocytes. By the use of inhibitors of the prenyl tranferase enzyme(s) responsible for prenylation, pharmacological inhibition of endothelial Rho protein function is effective in specifically controlling leukocyte recruitment to the CNS and subsequent neuroinflammatory disease.

The present invention therefore comprises the use of a protein prenyl transferase inhibitor for the alleviation of neuroinflammatory disease.

The present invention has been demonstrated with induced experimental autoimmune encephalomyelitis (EAE), the accepted animal model of multiple sclerosis (MS). It will be appreciated that the invention is applicable not only to the alleviation of MS but also to other diseases of the CNS including diseases of the eye e.g. uveitis.

The two principal types of prenylation of proteins in the present context are farnesyl and geranylgeranyl prenylation. Inhibition of either of these reactions will alleviate the disease to a significant extent but the best results have been obtained using a combination of a farnesyl transferase inhibitor and a geranylgeranyl transferase inhibitor. The combination of a farnesyl transferase inhibitor and a geranylgeranyl transferase inhibitor is a novel therapeutic product. In preparing medication for use in accordance with the invention the inhibitors may be combined in a single dosage form or as separate dosage forms in the same pack.

Two examples of inhibitor are the compounds known as FTI-277 and/or GGTI-298. These compounds have been described in the literature publications of Said M. Sebti which are well known to those working in this field. In particular the chemical structures of various FT and GGT inhibitors, including FTI-276, FTI-277, GGTI 297 and GGTI 298, are disclosed by T. F. McGuire et al, The Journal of Biological Chemistry, vol 271, No 44, November 1, pp 24702-24707, 1996. This publication also explains the process of prenylation and the different terminal peptide sequences to which the FT and GGT inhibitors attach. The contents of this prior publication are hereby incorporated in the disclosure of the present application. The inhibitors FTI-276, FTI-277 (and also GGTI-286 and GGTI-287) are commercially available from Calbiochem-Novabiochem Corporation, inter alia through their UK distributor CN BIOSCIENCES UK, Boulevard Industries Park, Padge Road, Nottingham, NG9 2JR.

The inhibitors may be formulated for parenteral administration. Dosages required for effective results will depend on the severity of the disease. By way of example, we recommend amounts of a farnesyl transferase inhibitor and/or a geranylgeranyl transferase inhibitor to provide for a daily dose of up to 25 mg or more per kg body weight e.g. from 10 to 25 mg/kg of each inhibitor used.

In the case of MS the patient should administer the medication when experiencing warning of an oncoming episode.

The research leading up to the present invention will now be described in detail.

### BACKGROUND

In order for T-lymphocytes to perform their immune function in tissue surveillance, they must be able to leave the circulation and traffic through the solid tissues of the body. Thus the migration of lymphocytes across the vascular endothelial cell (EC) wall is a prerequisite in the implementation of lymphocyte function. Lymphocyte transendothelial migration has been shown to be dependent on both lymphocyte activation (Pryce et al., 1997) and an ability to effectively elicit signalling responses in endothelial cells (Etienne et al, 1998; Adamson et al., 1999; Etienne et al., 2000). The recruitment and transvascular migration of T-lymphocytes to the CNS has been the subject of substantial investigation and has led to a greater understanding of the role of lymphocytes under normal and inflammatory conditions.

CNS endothelia, unlike endothelia from peripheral sites are connected together by impermeable tight junctions forming the blood-brain and inner blood-retinal barriers respectively (Rubin and Staddon 1999). Despite these cellular barriers, a low level of leukocyte traffic into the CNS occurs (Hickey et al, 1991) which can be dramatically up regulated during the development of immune-mediated diseases (Calder and Greenwood 1995). We have previously reported that ICAM-1 (CD54) is pivotal in mediating transendothelial migration of lymphocytes through the LFA-1/ICAM-1 (CD11a/CD54) interaction on CNS endothelial cells (Male et al., 1994; Greenwood et al, 1995). This dependence on the CD54/CD11a interaction results from intracellular signalling responses generated within CNS endothelial cells through the CD54 molecule which result in endothelial facilitation of transendothelial migration (Adamson et al, 1999).

We have previously demonstrated that efficient transduction of CD54 mediated signalling responses and consequently transendothelial migration of T-lymphocytes is critically dependent on functional EC Rho proteins (Adamson et al, 1999). Co-cultures of CNS endothelial cells with T-lymphocytes, or mimicking lymphocyte adhesion by cross-linking CD54 results in an increase in GTP-loaded endothelial Rho proteins (Adamson et al, 1999). In addition CD54 mediated signalling events and transendothelial lymphocyte migration are effectively inhibited following inactivation of endothelial Rho proteins with C3-transferase (Etienne et al, 1998; Adamson et al, 1999). Post-translational modification of Rho proteins, which result in their C-terminal prenylation, is essential for their correct subcellular localisation (Adamson et al, 1992a; 1992b) and function (Hori et al, 1991). C-terminal prenylation of Rho proteins occur on the cysteine residue which is 4 amino acids from the C-terminus, within a CAAX box motif (Adamson et al., 1992a). Both RhoA and RhoC are prenylated with a geranylgeranyl isoprenoid group whereas RhoB is prenylated by either geranylgeranyl or farnesyl isoprenoids. These prenylation reactions are catalysed by the protein prenyl transferase enzymes farnesyltransferase and geranygeranyltransferase type I (Seabra et al, 1991).

### SUMMARY OF THE INVENTION

We have now found that treatment of endothelial cells with inhibitors of protein prenyltransferases inhibits the in vitro migration of T-lymphocytes across CNS endothelial cell monolayers. Treatment of mice, which are susceptible to experimental autoimmune encephalomyelitis, with inhibitors of protein prenyltransferases results in reduced leukocyte recruitment to the CNS, which is accompanied by a significant attenuation of clinical disease, associated with leucocyte infiltration, in this animal model of multiple sclerosis.

### METHODS USED

Materials

[³H]-deoxy-glucose, horseradish peroxidase coupled rabbit anti-mouse IgG and ECL reagents were obtained from Amersham International (Bucks, UK). Polyclonal anti-Rho Ab was obtained from Autogenbioclear, UK. Unless otherwise stated all chemicals used were obtained from the Sigma Chemical Company (Dorset, UK).

Endothelial Cells

Rat CNS endothelial cells.

The immortalised Lewis rat brain endothelial cell line GP8/3.9 (Greenwood et al., 1996) was maintained in Ham's F-10 medium supplemented with 17.5% FCS, 7.5 μg/ml endothelial cell growth supplement, 80 μg/ml heparin, 2 mM glutamine, 0.5 μg/ml vitamin C, 100 U/ml penicillin and 100 μg/ml streptomycin.

Rat Aortic Endothelial Cells

Rat aortic endothelial cells were isolated by the method described by McGuire and Orkin (1987). Rat aorta was removed by dissection, cut into small pieces (2-5 mm) and placed luminal side down onto collagen-coated 24 well plates and cultured in RPMI supplemented with 20% foetal calf serum, 7.5 μg/ml endothelial cell growth supplement (Advanced Protein Products Ltd.), 80 μg/ml heparin, 2 mM glutamine, 0.5 μg/ml vitamin C, 100U/ml penicillin and 100 μg/ml streptomycin. After 3 days the explants were removed and outgrowing cells were expanded and passaged by trypsinisation. At confluence the cells had the “cobblestone” morphology characteristic of large vessel endothelium, expressed von Willebrand factor and grew in medium containing D-valine (a capacity lacking in fibroblasts and smooth muscle cells). Cells were used after passage 3, which is the earliest stage at which sufficient cells were available for experimentation.

MBP-specific CD4⁺ T Cell Lines

Lewis rat T-lymphocyte cell lines specific for purified myelin basic protein were prepared as previously described (Pryce et al, 1997). Briefly, lymph nodes were collected from bovine MBP-immunised rats and the T-lymphocytes propagated by periodically alternating antigen activation with IL-2 stimulation. The cell lines express the marker of the CD4⁺ T cell subset, are CD45RC^{low} and recognise MBP in the molecular context of MHC class II determinants (Pryce et al, 1997). These cells have previously been shown to be highly migratory across monolayers of primary cultured brain and retinal endothelia (Pryce et al, 1997) and represent antigen-stimulated lymphocytes.

Adhesion of Peripheral Lymph Node Cells to Endothelia

Adhesion assays were carried out as previously described (Pryce et. al., 1994; Adamson et al., 1999). Briefly, peripheral lymph node-derived cells (PLNC) were isolated and T-lymphocytes obtained after purification on nylon wool columns. These cells which represent non-antigen activated T-lymphocytes are therefore non-migratory but highly adhesive when activated with the mitogen concanavalin A (Pryce et al., 1994, Greenwood and Calder 1993; Male et al., 1994 ). PLNC were activated for 24 h with type V concanavalin A, washed twice in HBSS, and cells labelled with 3 μCi [³H]-deoxy glucose per 10⁶ cells in HBSS for 90 min at 37° C. After washing the cells three times with HBSS they were resuspended in RPMI 1640 medium containing 10% FCS. Endothelial monolayers grown on 96 well plates were prepared by removing the culture medium and washing the cells four times with HBSS. 200 μl of [³H]-labelled PLNC at a concentration of 1×10⁶/ml were then added to each well and incubated at 37° C. for 1.5 h. In each assay, β-emissions from each of 6 replicate blank wells were determined to provide a value for the total amount of radioactivity added per well and to allow calculation of the specific activity of the cells. After incubation, non-adherent cells were removed with four separate washes from the four poles of the well with 37° C. HBSS as previously described (Pryce et al, 1994: Adamson et al., 1999). Adherent PLNC were lysed with 2% SDS, the lysate removed, scintillant added and radioactivity quantitated by spectrometry. Results are expressed as the means±SEM between groups determined by Student's t-test.

T-Lymphocyte Transendothelial Migration

The ability of the immortalised cells to support the transendothelial migration of antigen specific T-lymphocytes was determined using a well-characterised assay as described extensively elsewhere (Adamson et al., 1999 and http://www.ucl.ac.uk/ioo/video/ sequence.avi) Briefly, T-lymphocytes were added (2×10⁵ cells/well) to 24 well plates containing endothelial cell monolayers. Lymphocytes were allowed to settle and migrate over a 4 h period. To evaluate the level of migration co-cultures were placed on the stage of a phase-contrast inverted microscope housed in a temperature controlled (37° C.), 5% CO₂ gassed chamber (Zeiss, Herts, U.K.). A 200×200 μm field was randomly chosen and recorded for 10 min spanning the 4 h time point using a camera linked to a time-lapse video recorder. Recordings were replayed at 160× normal speed and lymphocytes identified and counted which had either adhered to the surface of the monolayer or that had migrated through the monolayer. Lymphocytes on the surface of the monolayer were identified by their highly refractive morphology (phase-bright) and rounded or partially spread appearance. In contrast cells that had migrated through the monolayer were phase-dark, highly attenuated and were seen to probe under the endothelial cells in a distinctive manner (Pryce et al, 1997; Adamson et al, 1999: Etienne et al, 2000). All other data were expressed as a percentage of the control migrations. A minimum of three independent experiments using a minimum of 6 wells per assay were performed. The results are expressed as the means±SEM and significant differences between groups determined by Student±s t-test.

Preparation of Plasma Membranes and Western Blotting

Ice-cold lysis buffer containing 10 mM Tris-HCl pH7.5, 5 mM MgCl₂, 1 mM DTT and 1 mM PMSF was added to cells and incubated on ice for 10 min. Cells were subsequently homogenised and centrifuged at 5000 g for 10 min to remove nuclei. Supernatants were then centrifuged at 100,000 g in a Beckman Ultracentrifuge for 30 min to obtain crude membranes. Membrane pellets were washed with buffer containing 50 mM Tris-HCl pH7.5, 50 mM NaCl, 5 mM MgCl₂, 1 mM DTT and 1 mM PMSF and re-centrifuged at 100,000 g for 30 min. Membrane pellets were then resuspended in sample buffer and proteins resolved on 12.5% SDS-PAGE gels. Proteins were electroblotted on nitrocellulose membranes and immunblotted with anti-Rho polyclonal antibody (Santa Cruz, Wilts, UK). Rho proteins within membrane fractions were visualised following incubation with a 1:15,000 dilution of goat anti-rabbit-HRP (Pierce, Chester, UK) and ECL development (Amersham, Bucks,UK).

Induction of Treatment of EAE

6-8 week Biozzi ABH mice were from stock purchased from Harlan Olac (Bicester, UK), these were maintained on RM-1(E) diet and water ad libitum. These were injected with 1 mg of syngeneic spinal cord homogenate in Freund's adjuvant on day 0 & 7 (Baker et al 1990). Animals were checked daily and scored 0=normal, 1=limp tail, 2=impaired righting reflex, 3=paral paralysis, 4=complete hindlimb paralysis. (Baker et al 1990, O'Neill et al 1991). Animals were injected daily i.p. with 0.1 ml of vehicle or inhibitors dissolved in PBS containing 50% DMSO. Animals were killed and brains and spinal cords were removed and either snap-frozen in liquid nitrogen for immunocytochemistry or were fixed in formal saline, embedded in paraffin wax, sectioned and stained with hematoxylin and eosin. Cryostat sections were stained using an indirect immunoperoxidase technique for CD3, CD4, CD8, CD11b, CD54, CD106 as described previously (Baker et al 1990). Differences between groups were assessed using Mann Whitney U non parametric statistics.

### RESULTS

Treatment of Brain Endothelial Cells with the Protein Prenyl Transferase Inhibitors FTI-277 and GGTI-298 Prevents Rho Protein Association with Cell Membranes.

In order to establish the length of treatment with protein prenyl transferase inhibitors necessary to prevent the majority of Rho protein being prenylated, and hence inactivated, whole cell membranes were prepared from control and treated brain endothelial cells by high-speed centrifugation. Isoprenylation of Rho proteins are essential for their efficient association with cell membranes (Adamson et al, 1992). Western blot analysis of control brain endothelial cell membranes showed that Rho proteins were associated with cell membranes. Following treatment with 10 μM FTI-277 (Lerner et al, 1995) and 10 μM GGTI-298 (McGuire et al, 1996) for 48 h there was a significant reduction in the amount of Rho protein associated with the cell membrane fraction derived from CNS endothelial cells (FIG. 1A). This effect was not observed when the endothelial cells were treated for 24 h which suggests that 48 h pre-treatment with protein prenyl transferase inhibitors is required to fully prevent the prenylation of cellular Rho proteins and their subsequent localisation at cell membranes. Conversely, inactivation of endothelial Rho proteins through ADP-ribosylation with C3-transferase is independent of protein prenylation and thus did not affect the subcellular distribution of Rho proteins (FIG. 1A).

Treatment of aortic endothelial cells with 10 μM FTI-277 and 10 μM GGTI-298 for 48 h in an identical manner to that described above for brain endothelia also resulted in a marked reduction in membrane associated Rho proteins (FIG. 1B ).

Treatment of Endothelial Cells with Inhibitors of Protein Prenyl Transferase Inhibits T-Lymphocyte Migration through Brain but Not Aortic Endothelial Cell Monolayers.

Rat endothelial cell monolayers derived from brain and aorta were able to support the transendothelial migration of antigen-specific T-lymphocytes over a 4 h period with 43.0±4.6% and 31.4±5.4% of the lymphocytes migrating through the endothelial cell monolayers respectively. Treatment of brain endothelial cell monolayers with 10 μM FTI-277 for 24 h prior to, and during the 4 h T-lymphocyte co-culture, did not result in a significant alteration in T-cell migration through the endothelial cell monolayer. However, under identical conditions treatment with 10 μM GGTI-298 resulted in a significant inhibition of transendothelial lymphocyte migration to 73.5±6.0% of control migration (P<0.005 verses controls, n=30) (FIG. 2A). When 10 μM FTI-277 was used in combination with 10 μM GGTI-298, this resulted in a further inhibition of lymphocyte migration to 60.4±5.1% of control migration (P<0.005 verses controls, n=30) (FIG. 2A). This level of inhibition did not approach that achieved with C3 transferase which inhibited T-cell migration to 18.4±4.1% of control values (P<0.005 verses controls, n=12).

Increasing the time brain endothelial cells were exposed to protein prenyltransferase inhibitors from 24 to 48 h and continuing their presence during the 4 h T-lymphocyte co-culture, resulted in a greater reduction in T-cell migration. Treatment of the endothelial cell monolayer with 10 μM FTI-277 reduced migration to 77.7±4.9% of control migration (P<0.005 verses controls, n=30) and 10 μM GGTI-298 to 51.6±3.1% of control migration (P<0.005 verses control, n=30 and P<0.005 verses the 24 h treated animals) (FIG. 2B). A combination of both FTI-277 and GGTI-298 resulted in a further reduction of T-cell migration to 39.3±6.4% of controls (P<0.005 verses controls, n=30 and P<0.02 verses 24 h treated animals) (FIG. 2B). This temporal observation is consistent with the demonstration that inhibition of Rho protein prenylation required 48 h pre-treatment to prevent its association with membrane fractions. The degree of inhibition of T-cell migration with combined FTI-277/GGTI-298 treatment approached that obtained following C3-transferase treatment of endothelial cells which results in an inhibition of transendothelial lymphocyte migration to 18.4±4.1% of control value (P<0.005 verses controls, n=12).

Non of the observed inhibitory effects on migration were due to the prenyltransferase inhibitors affecting the T cells during the 4 h coculture as the presence of the inhibitor during a 4 h coculture alone had no effect on migration (data not shown). Furthermore, treatment of the MBP T-cell line for a total of 52 h (48 h pre-treatment plus 4 h coculture with EC) with a combination of 10 μM FTI-277 and 10 μM GGTI-298 brought about only a small reduction in T cell migration to 87.4±2.8% of control migration (vehicle treated T cells=100.7±1.9%) and did not approach the level of inhibition achieved when the EC monolayer was treated with the inhibitors for the same duration.

Treatment of brain endothelia with either FTI-277 or GGTI-298 for 24 or 48 h, plus the 90 min adhesion assay, did not have any significant effect on the ability of T-lymphocytes to adhere to brain endothelial cells. However, a combination of both 10 μM FTI-277 and 10 μM GGTI-298 under identical conditions resulted in small but significant reduction in T-lymphocyte adhesion to brain endothelial cells following both 24 and 48 h treatments (FIGS. 2A, B).

The finding that treatment of the GP8/3.9 brain endothelial cell line with protein prenyl transferase inhibitors is effective in causing a significant reduction in the transendothelial migration of T-lymphocytes, but not their adhesion, demonstrates that this effect is predominantly due to the inhibition of endothelial cell support of lymphocyte migration. Contrary to the findings with brain endothelial cells, the treatment of aortic endothelial cells with a combination of 10 μM FTI-277 and 10 μM GGTI-298 for 48 h and during the T-cell co-culture did not have any affect on either T-lymphocyte adhesion or migration (FIG. 2C). The inability of protein prenyltransferase inhibitors to significantly attenuate T-lymphocyte migration through aortic endothelial cell cultures suggests that Rho proteins are not functionally important in aortic EC for facilitating lymphocyte migration.

Treatment of Biozzi ABH Mice with a Combination of Protein Prenyl Transferase Inhibitors Attenuate the Clinical Signs of EAE.

Biozzi ABH mice induced with EAE began to show clinical signs of disease 13 days after initial inoculation with syngeneic spinal cord homogenate with the peak of disease occurring at day 17 (FIG. 3A). Of a total of 15 positive EAE control animals, 13 developed disease of which 2 animals progressed to grade 3 disease (partial hind limb paralysis) and 11 progressed to grade 4 (complete hind limb paralysis) (Table 1). The remaining 2 animals showed no observable signs of disease. The mean clinical score for the whole group was 3.2±0.4 (n=15) and of the animals that developed disease 3.7±0.1 (n=13) with a mean day of disease onset of 15.1±1.2 days. Vehicle treated animals (n=16) showed a disease progression that was similar to untreated controls. Of the 16 animals, 13 showed clinical signs of EAE with 3 animals displaying symptoms of grade 3 disease and 10 progressing to grade 4 disease. The remaining 3 animals in this group showed no signs of EAE. The mean clinical disease score for the whole group of vehicle treated animals was 3.0±0.4 (n=16) and for those animals displaying disease was 3.7±0.2 with onset appearing as early as day 13 with mean onset of disease being at 15.0±1.5 days (FIG. 3A; Table 1). Neither of these disease indices were significantly different from untreated EAE control animals.

Animals which had been treated from day 9 to day 24 with a combination of 25 mg/kg FTI-277 and 25 mg/kg GGTI-298 showed grade 4 disease in only 2 of the 13 animals with one animal developing grade 3 disease, one animal showing grade 2 disease (impaired righting reflex) and a further 2 animals with grade 1 disease (limp tail). Seven of the13 animals which were treated with combination prenyl transferase inhibitors showed no signs of clinical disease. The mean group score of clinical disease was 1.3±0.5 (n=13) which was significantly different from both untreated control EAE animals (P<0.01) and vehicle treated control EAE animals (P<0.05). Of the animals which developed disease in this group, the mean disease index was 2.4±0.6 (n=6) which was also significantly different from untreated control EAE animals (P<0.05) (FIG. 3A; Table 1). The onset of disease in treated animals was unchanged at 15.3±0.8 days.

Prior Treatment of Biozzi ABH Mice with a Combination of Protein Prenyl Transferase Inhibitors does not Prevent Subsequent Rapid Induction of Disease Following Antigen Challenge

Biozzi ABH mice, which were induced for EAE but showed no signs of disease following treatment with the combined protein prenyltransferase inhibitors, or control animals in which active disease had remitted after day 30, were re-innoculated with spinal cord homogenate in complete Freund's adjuvant at day 68. Animals were selected that had not relapsed spontaneously. Both EAE control animals (n=7) and protein prenyl transferase inhibitor pre-treated animals (n=6) developed severe disease relapse equating to a mean disease score of 3.6±0.1 and 3.8±0.1 respectively. Disease induction was rapid in both groups with mean day of onset being 8.0±1.5 and 8.9±2.2 days post re-inoculation respectively (FIG. 3B). Neither mean group clinical disease score nor mean day of onset was significantly different between the two groups (Student's t test).

Treatment of Biozzi ABH Mice with a Combination of Protein Prenyl Transferase Inhibitors Restricts Leukocyte Migration into the CNS.

Biozzi ABH mice were sacrificed for histology and immunohistochemical analysis on day 19 post inoculation, during which clinical disease is normally at its height (Brennan et al, 1999). Sections of spinal cord, cerebrum and cerebellum were examined from control EAE animals (n=5) and combined protein prenyl transferase inhibitor treated EAE animals (n=6). In EAE control animals (all grade 4 disease) characteristic lesions were observed around vessels that consisted of substantial perivascular cuffing of leukocytes and infiltration into the parenchyma of the spinal cord. Examination of the cerebrum and cerebellum also revealed leukocyte cuffing of vessels although to a much lesser extent. Similar observations were seen with the vehicle treated animals. In contrast, animals which were treated daily between day 9 and day 19 with a combination of 25 mg/kg FTI-277 and 25 mg/kg GGTI-298 (grade 0 disease) showed no evidence of perivascular cuffing of leukocytes or infiltration into the parenchyma in any of the tissues examined (FIG. 4A panels a-h). In contrast to control groups, immunohistochemical analysis of spinal cord sections demonstrated the absence of both T-lymphocytes and macrophages in protein prenyltransferase treated animals (FIG. 4B panels i-l).

### CONCLUSIONS

We have previously shown that T-lymphocyte migration through CNS endothelial cell monolayers initiates increased Rho-GTP loading and is essential for T-lymphocyte migration through monolayers of CNS endothelial cells (Adamson et al, 1999). Treatment of endothelial cells with C3-transferase, which is able to covalently modify and inhibit the classical Rho proteins A, B and C (Aktories et al, 1989) is effective in inhibiting both Rho-GTP loading and transendothelial lymphocyte migration (Adamson et al, 1999). In addition, other signalling cascades induced through leukocyte adhesion to endothelial cells are also inhibited following treatment of endothelial cells with C3-transferase (Etienne at al, 1998). These observations suggest that cellular Rho proteins are important in orchestrating the endothelial response to T-lymphocyte adhesion, which subsequently results in their transendothelial migration. In order to be functional Rho proteins undergo a series of post-translational C-terminal modifications which are initiated through the addition of a isoprenoid group to the C-terminal cysteine residue (Adamson et al, 1992). It has previously been determined that both RhoA and RhoC are substrates for protein geranylgeranyltransferase type I (GGTase I) which catalyses the addition of geranylgeranyl group to the Rho C-terminus (Katayama et al, 1991). RhoB has been proposed to exist in two distinct forms, resulting in cellular populations in which are either geranylgeranylated or farnesylated (Adamson et al, 1992). The farnesylation and geranylgeranylation of RhoB both appears to be catalysed by GGTase I (Armstrong et al, 1996) and it has been observed that inhibition of protein farnesylation of RhoB results in increased levels of geranylgeranylated RhoB (Lebowitz et al, 1997). Such protein prenylation of Rho proteins is essential for effective targeting to cellular membranes (Adamson et al, 1992) and interaction with specific effector molecules (Hori et al, 1991). The CAAX box peptidomimetic protein prenyl transferase inhibitors FTI-277, which is effective in blocking protein farnesylation of Rho proteins and GGTI-298 which effectively blocks protein geranylgeranylation, both showed some activity in attenuating T-lymphocyte migration through monolayers of CNS derived endothelial cells. However their inhibitory effect was increased when endothelial cells were exposed to a combination of inhibitors suggesting that all prenylated forms of Rho may be involved in the endothelial cell response to leukocyte adhesion. The effectiveness of protein prenyl transferase inhibitors in inhibiting endothelial support of T-lymphocyte migration was greatest when endothelial cells were exposed to inhibitors for 48 hrs prior to co-culture with T-lymphocytes and when inhibitor concentrations were maintained during the period of endothelial/T-lymphocyte co-culture. Rho proteins undergo cycles of prenylation and de-prenylation with specific half lives of the prenylated form. It has previously been determined that the half life of RhoA prenylation is in the order of 31 hours (Backlund 1997) which therefore correlates well with the increased ability of protein prenyltransferase inhibitors to affect transendothelial lymphocyte migration following 48 hrs pre-treatment. However RhoB is an immediate early gene (Jahner and Hunter, 1991), and ICAM-1 cross-linking which mimics leukocyte adhesion to endothelial cells (Etienne et al, 1998; Adamson et al, 1999) results in the rapid induction of RhoB mRNA (unpublished observations). In keeping with its role as an immediate early gene the half life of this protein is between 2-4 hrs (Lebowitz et al, 1995). This would therefore necessitate the continued presence of protein prenyltransferase inhibitors during the 4 hr co-culture of endothelial cells and lymphocytes in order to prevent the effective prenylation on newly synthesised RhoB. Interestingly pre-treatment of aortic endothelial cells with combination of protein prenyl transferases was ineffective in attenuating transendothelial lymphocyte migration suggesting that the effects of these agents are specific to CNS endothelial cells and demonstrating heterogeneity in endothelia derived from different sites. These studies also suggest that lymphocyte migration into the brain paranchyma is more regulated than into other tissues and may therefore explain the lower level of leukocyte infiltration into the CNS under normal conditions. The ability of T-lymphocytes to migrate through aortic endothelial cell monolayers also demonstrate that T-lymphocytes are not affected by the presence of protein prenyltransferase inhibitors during the co-culture period.

Experimental allergic encephalomyelitis (EAE) is a chronic relapsing and remitting mouse model of multiple sclerosis, which is induced following inoculation of susceptible animals with syngeneic spinal cord homogenate (Baker et al, 1990) and is dependent on the infiltration of leucocytes into the brain and spinal cord (Brennen et al, 1999). Leucocyte recruitment to the CNS occurs at a precise time prior to the development of clinical signs and inhibition of such recruitment to the CNS is sufficient to inhibit subsequent disease progression (Butter et al, 1991; Allen et al, 1993). Combination treatment of biozzi ABH mice with both farnesyltransferase and geranylgeranyltransferase inhibitors were able to significantly attenuate both the infiltration of leukocytes into the CNS of biozzi ABH mice and alleviate the clinical signs of disease. Therapy with protein prenyltransferase inhibitors was commenced at day 9 following initial inoculation of animals with antigen since it has previously been shown that treatment of mice with daily injections of 25 mg/kg. FTI-277 i.p. required 3 days to obtain steady state plasma concentrations and infiltration of leukocytes into the CNS normally occurs at around day 12 (Brennan et al, 19990). This treatment regimen was able to dramatically reduce both the number of animals showing clinical signs of EAE and the severity of the disease without delaying the onset of disease. Animals in which combination therapy had stopped after day 24, as well as control animals showed full remission of disease at day 30. It was interesting to note that a further challenge of animals with spinal cord homogenate at day 68, in control and animals previously treated with protein prenyltransferase inhibitors up to day 24 subsequently developed severe disease. The rapid re-induction of disease in these animals demonstrates that the normal disease producing mechanisms and sensitisation to spinal cord homogenate antigen are not affected during treatment of animals with protein prenyltransferase inhibitors suggesting these agents effective in inhibiting leukocyte migration to the CNS. The precise mechanism by which Rho proteins may mediate transvascular migration of leukocytes is currently unknown.

A number of studies have attempted to control leukocyte trafficking by targeting both leukocyte-endothelial cell adhesion and intracellular signalling pathways. Antibodies directed against α₄β₁ EAE (Yednock et al, 1992) and α₄-integrin (Kent et al, 1995) have been shown to inhibit the clinical signs of EAE. The use of anti-α₄ integrin antibody treatment in multiple sclerosis patients with secondary progressive disease has demonstrated a marked improvement in the incidence of new lesions (Turbridy et al, 2000). The effectiveness of these agents in both mouse and human studies suggest that observations in mouse EAE studies can be accurately extrapolated to human neuroinflammatory disease. Studies aimed at affecting intracellular signalling pathways have used general inhibitors of tyrosine kinases which were also found to alleviate the clinical signs of EAE (Constantin et al, 1998;1999) although whether these agents are affecting leukocytes or vascular endothelial cells was not clear from these studies.

It is envisaged that additional brain pathologies which have a significant inflammatory component such as neuroAIDS and Alzheimers disease will be significantly alleviated following treatment with these inhibitors.

### REFERENCES

Adamson P., Etienne S., Couraud P-O., Calder V. and Greenwood J. (1999) T-lymphocyte migration through CNS endothelial cells involves signalling through endothelial ICAM-1 via a rho dependent pathway. J. Immunol. 162: 2964-2973

Pryce, G., D. K. Male, I. Campbell, and J. Greenwood. 1997. Factors controlling T-cell migration across rat cerebral endothelium in vitro. J. Neuroimmunol. 75:84.

Hickey, W. F., B. L. Hsu, and H. Kimura. 1991. T-lymphocyte entry into the central nervous system. J. Neurosci. Res. 28:254.

Greenwood, J., and V. Calder. 1993. Lymphocyte migration through cultured endothelial cell monolayers derived from the blood-retinal barrier. Immmunology. 80:401.

Osborn, L. 1990. Leukocyte adhesion to endothelium in inflammation. Cell 62:3.

Springer, T. A. 1994. Traffic signals for lymphocyte recirculation and leukocyte emigration: the multistep paradigm. Cell 76:301.

Yednock, T .A., C. Cannon, I. C. Fritz, F. Sanchez-Madrid, L. Steinman, and N. Karin. 1992. Prevention of experimental autoimmune encephalitis by antibodies against α₄β₁ integrin. Nature 356:63.

Male, D., J. Rahman, G. Pryce, T. Tamatani, and M. Miyasaka. 1994. Lymphocyte migration into the CNS modeled in vitro: roles of LFA-1, ICAM-1 and VLA-4. Immunology 81:366.

Greenwood, J., Y. Wang, and V. Calder. 1995. Lymphocyte adhesion and transendothelial migration in the CNS: the role of LFA-1, ICAM-1, VLA-4 and VCAM-1. Immunology 86:408.

Greenwood, J., G. Pryce, L. Devine, D. K. Male, W. L. C. dos Santos, V. L. Calder, and P. Adamson. 1996. SV40 large T immortalised cell lines of the rat blood-brain and blood-retinal barriers retain their phenotypic and immunological characteristics. J. Neuroimmunol. 71:51.

Etienne S., P. Adamson, J. Greenwood, A. D. Strosberg, S. Cazaubon, and P-O. Couraud. 1998. Rho-dependent signaling pathways coupled to ICAM-1 in microvascular brain endothelial cells. J. Immunol. In press

Greenwood, J. 1992. Characterisation of a rat retinal endothelial cell culture and the expression of P-glycoprotein in brain and retinal endothelium. J. Neuroimmunol. 39:123.

Male, D., G. Pryce, C. Hughes, and P. Lantos. 1990. Lymphocyte migration into brain modeled in vitro: control by lymphocyte activation, cytokines, and antigen. Cell. Immunol. 127:1.

Jahner, D., and T. Hunter. 1991. The ras related gene rhoB is an immediate early gene inducible by v-fps, epidermal growth factor and platelet derived growth factor in rat fibroblasts. Mol. Cell. Biol. 11:3682.

Aktories, K., S. Braun, S. Rosener, I. Just, and A. Hall. 1989. The rho gene product expressed in E.Coli is a substrate for botulinum ADP-ribosyltransferase C3. Biochem. Biophys. Res. Commun. 158:209.

Adamson, P., Marshall, C. J. Hall, A. and Tilbrook, P. A. (1992). Post-translational modifications of the p21^{rho} proteins. J. Biol. Chem. 267: 20033.

Adamson P., Paterson, H. F. and Hall, A. (1992). Intracellular localisation of the p21^{rho} proteins. J. Cell. Biol. 119: 617.

Baker, D., O'Neill, J. K., Wilcox, C., Gschmeissner, S., Butter, C. and Turk, J. L. (1990). Induction of chronic relapsing experimental allergic encephalomyelitis in Biozzi mice. J. Neuroimmunol 28: 261.

Brennan, F. R., O'Neill, J. K., Allen, S. J. Butter, C., Nuki, G. and Baker, D. (1999). CD44 antigen is involved in selective leukocyte extravasation during inflammatory central nervous system disease. Immunology. 98: 427.

Norgaard P., Law, B., Joseph, H., Page, D. L., Shyr, Y., Mays, D., Pitenpol, J. A., Kohl, N., Oliff, A., Coffey, R. J., Poulsen, H S. and Moses, H. L. (1999). Treatment with farnesyl protein transfer inhibitor induces regression of mammary tumors in transforming growth factor (TGF) and TGFα/neutransgenic mice by inhibition of mitogenic activity and induction of apotosis. Clin Cancer Res. 5: 35.

Constantin, G., Brocke, S., Izikson, A., Laudana, C, and Butcher, E. C. (1998). Tyrphostin AG490, a tyrosine kinase inhibitor blocks actively induced experimental autoimmune encephalomyelitis. Eur. J. Immunol. 28: 3523.

Constantin, G., Laudana, C., Brocke, S. and Butchr, E. C. (1999). Inhibition of Experimental Autoimmune Encephalomyelitis by a tyrosine kinase inhibitor. J. Immunol. 162: 1144.

Butter, C., Baker, D., O'Neill, J. K. and Turk, J. L. (1991). Mononuclear cell traffic and plasma protein extravasation into the CNS during chronic relapsing experimental allergic encephalomyelitis in Biozzi AB/H mice. J. Neurol Sci, 104: 9.

Allen, S. J., Baker, D., O'Neill, J. K., Davison, A. N. and Turk, J. L. (1993). Kinetics of cellular infiltration of the central nervous system during chronic relapsing experimental allergic encephalomyelitis in the Biozzi AB/H mouse. Cell. Immunol. 146: 335.

Tubridy, N., Behan, P. O., Capildeo, R., Choudhuri, A., Forbes, R., Hawkins, C. P., Hughes, R. A. C., Palace, J., Sharrack, B., Swingler, R., Young, O. C., Moseley, I. F., MacManus, D. G., Donaghue, S. Miller, D. H and the UK integrin study group. (2000). The effect of anti-alpha 4 integrin antibody on brain lesion activity in MS. Neurology 53: 466.

Calder, V. and Greenwood, J. (1995). Role of the vascular endothelium in immunologically-mediated neurological disease. In: Immunological aspects of the vascular endothelium. C. Savage and J. Pearson (Eds), Cambridge University Press. pp 96-123.

Rubin, L. L. and Staddon, J. M. (1999). The cell biology of the blood-brain barrier. Annu. Rev. Neurosci. 22: 11-28.

Hori Y., Kikuchi A., Isomura M., Katayama M., Miura Y., Fujioka H., Kaibuchi K. and Takai Y. (1991) Post-translational modifications of the C-terminal region of the rho protein are important for its interaction with membranes and the stimulatory and inhibitory GDP/GTP exchange proteins. Oncogene 6: 515-522

Lebowitz P., Case P. J., Prendergast G. C. and Thissen J. A. (1997) Farnesyltransferase inhibitors alter the prenylation and growth-stimulating function of RhoB. J. Biol. Chem. 272: 15591-15994.

Katayama M., Kawata M., Yoshida Y., Horiuch H., Yamamato K and Takai Y. (1991) The posttranslationally modified C-terminal structure of bovine aortic smooth muscle rhoA p21.J. Biol. Chem. 266: 16981-16984

Armstrong S. A., Hannah V. C., Goldstein J. L. and Brown M. S.(1995) CAAX geranygeranyl transferase, transfers farnesyl as effeciantly as geranylgeranyl to RhoB. J. B. C. 270: 26059-26062.

Kohl N. E., Omer A. C., Conner M. W., Anthony N. J., Davide J. P., DeSolms S. J., Giuliani E. A., Gomez R. P., Graham S. L., Hamilton K., Handt L. K., Hartman G. D., Koblan K. S., Kral A. M., Miller P. J., Mosser S. D., O'Neill T. J., Rands E., Schaber M. D., Gibbs J. B. and Oliff A. (1996) Inhibition of farnesyltransferase induces regression of mammary and salivary carcinomas in ras transgenic mice. Nature Medicine 1: 792-797.

Backlund P. S. (1997) Post-translational processing of RhoA. J. Biol. chem. 272: 33175-33180.

Lebowitz P. F., Davide J. P. and Prendergast G. C. (1995) evidence that farnesyltransferase inhibitors supress ras transformation by interfearing with Rho activity. Mol. Cell. Biol. 15: 6613-6622

Mangues R., Corral T., Kohl N. E., Symmans W. F., Lu S., Malumbres M., Gibbs J. B., Oliff A and Pellicer A. (1998) Antitumor effect of a farnesyl protein transferase inhibitor in mammary and lymphoid tumors overexpressing N-ras in transgenic mice. Cancer Res 58: 1253-1259

### LEGENDS TO FIGURES

Table 1: Treatment of Biozzi ABH Mice with a Combination of Protein Prenyl Transferase Inhibitors Inhibits EAE Disease Induction.

EAE was induced following subcutaneous inoculation into the flank of mice with 1 mg of spinal cord homogenate in complete Freund's adjuvant at day 0 and day 7. Animals were monitored daily for signs of clinical disease. Protein prenyl transferase inhibitors FTI-276 (25 mg/kg) and GGTI-297 (25 mg/kg) were given daily by intraperitoneal injection from day 9 to day 24. Animals were scored on day 18 post-innoculation as follows: no signs of disease were scored as 0 whereas animals displaying a limp tail, impaired righting reflex, partial hind limb paralysis, or complete hind limb paralysis were scored from 1-4 respectively.

FIG. 1: Treatment with FTI-277 and GGTI-298 Prevents Membrane Association of Rho Proteins in Brain Endothelial Cells.

Brain endothelial cells were treated with both FTI-277 and GGTI-298 for 24 or 48 h or C3-transferase. Membrane proteins resolved on 12.5% SDS-PAGE. Proteins were transferred to nitrocellulose membranes and immunoblotted with rabbit anti-Rho antibody.

FIG. 2: Effect of Endothelial Pre-Treatment with Protein Prenyl Transferase Inhibitors on Lymphocyte Adhesion to, and Migration through, Brain and Aortic Endothelial Cell Monolayers.

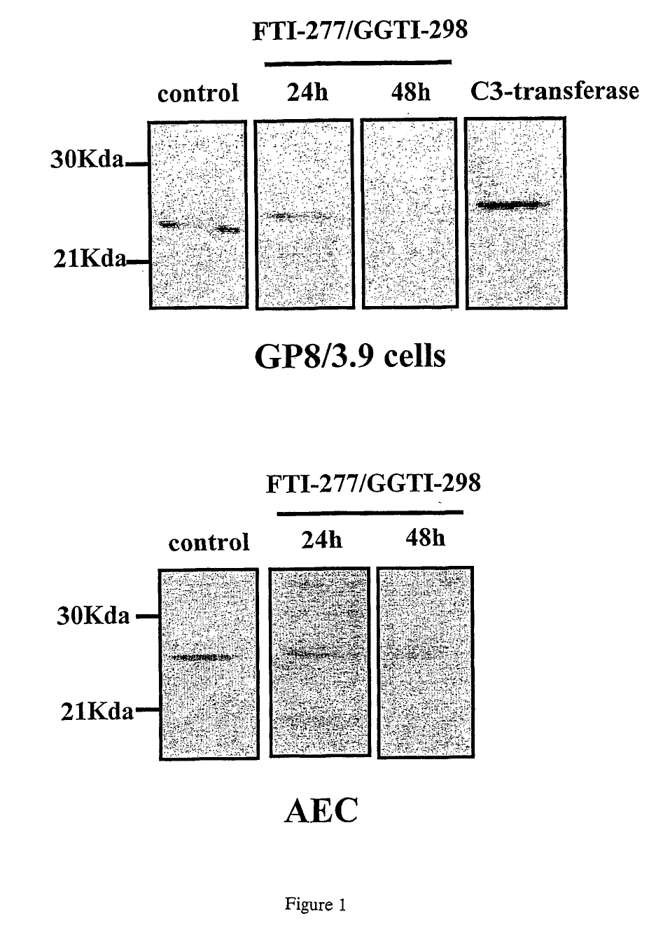

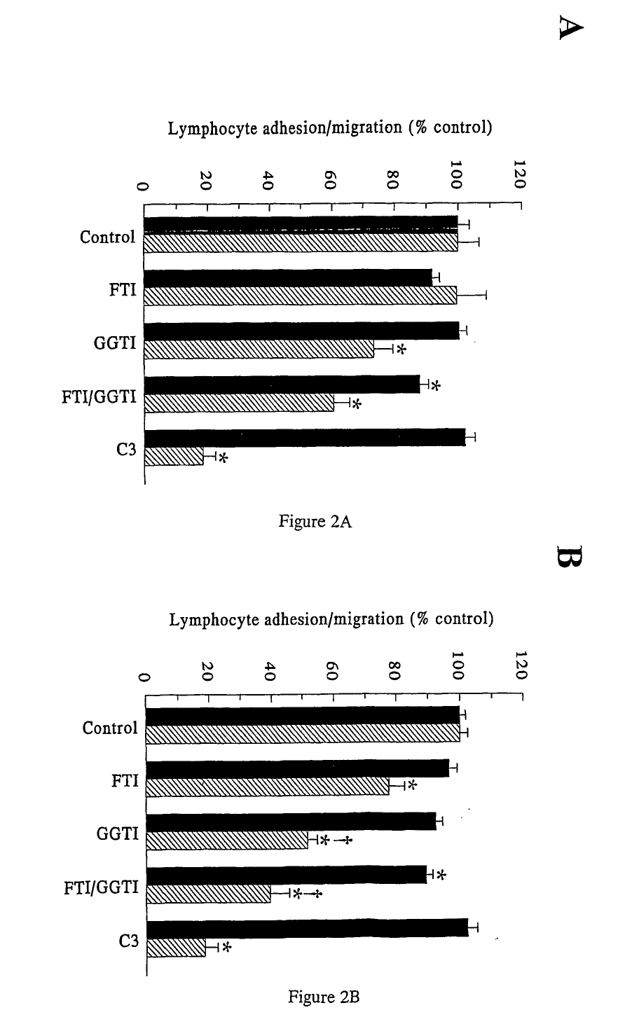

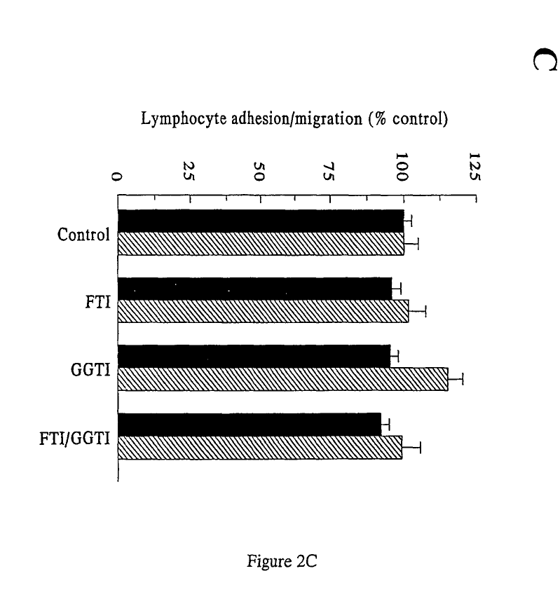

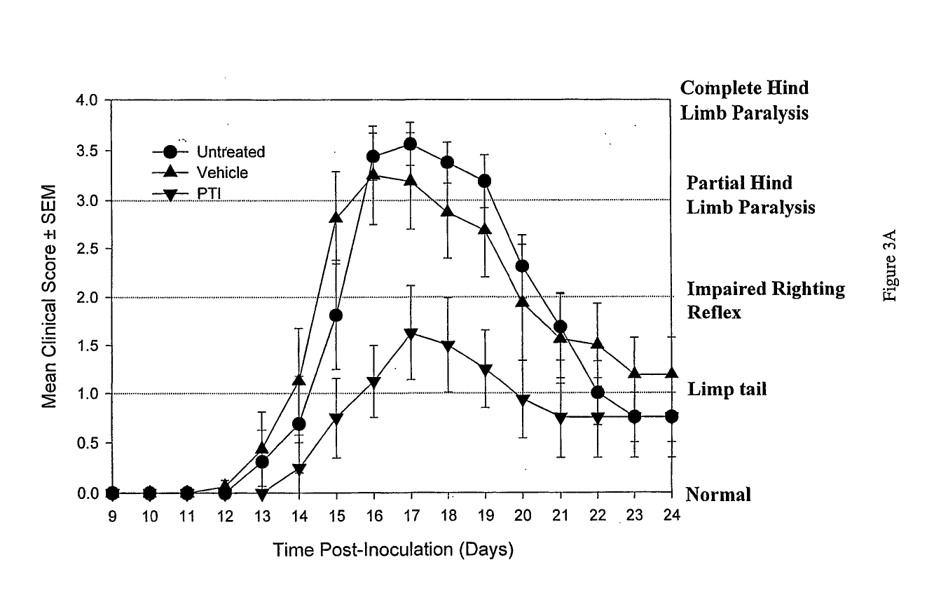

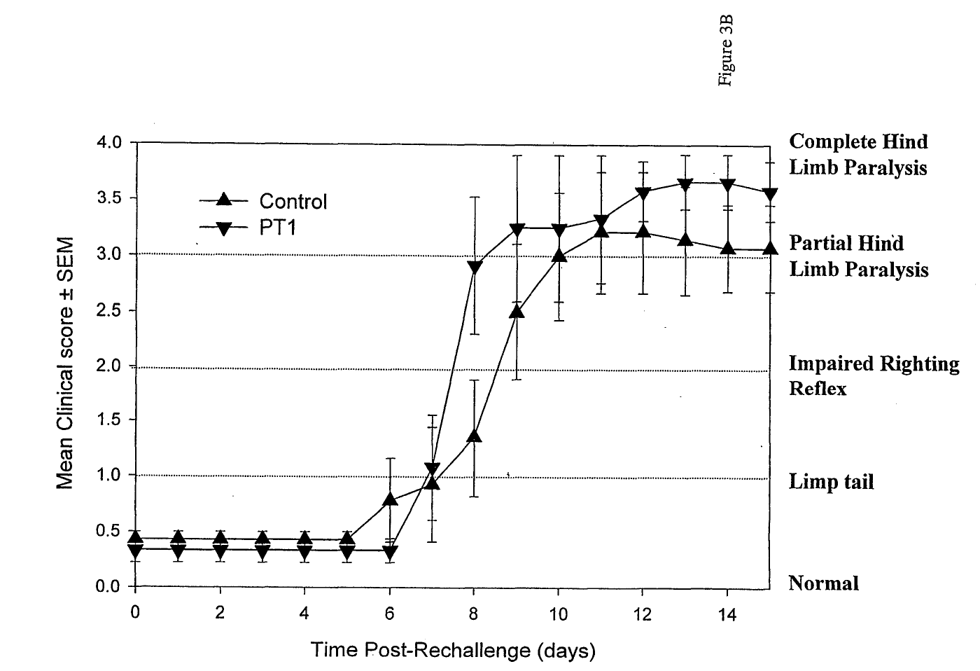

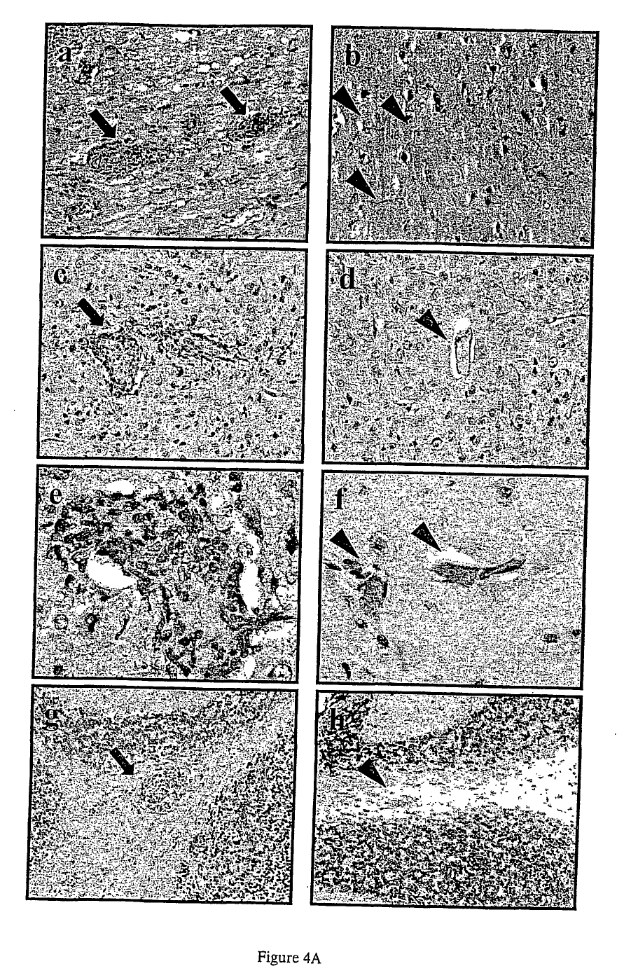

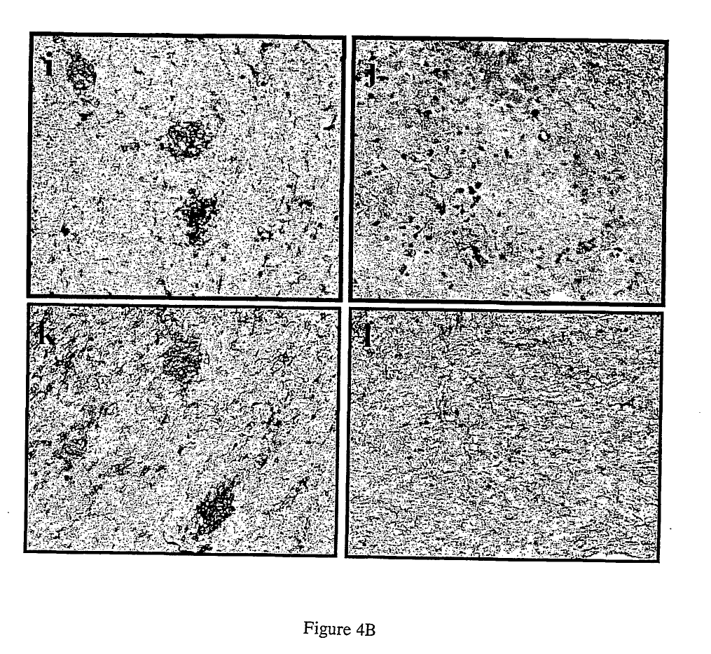

Transendothelial migration of MBP-specific T-lymphocytes (solid bars) and adhesion of mitogen activated rat peripheral lymph node lymphocytes (hatched bars) to brain and aortic endothelial cells was evaluated as described in detail in the text. (A) Brain endothelial cells pre-treated for 24 h with the protein prenyl transferase inhibitors FTI-277 (FTI), GGTI-298 (GGTI), FTI/GGTI or with C3 transferase prior to co-culture with lymphocytes. Protein prenyl transferase inhibitors were maintained during incubation with lymphocytes. (B) Brain endothelial cells pre-treated for 48 h with FTI, GGTI and FTI/GGTI combination as described above. (C) Cultures of rat aortic endothelial cells were pre-treated for 48 h with FTI, GGTI and FTI/GGTI combination and the inhibitors were maintained in the media during T-cell co-culture. In all cases observations are a minimum of three independent experiments using 10 wells per assay for the FTI-277/GGTI-298 inhibitors and 4 wells per assay for C3-transferase treatment. Data is expressed as mean+/−SEM percent of control migration. Significant differences between groups were determined by Student's t test.*P<0.005.

FIG. 3: Treatment of Biozzi ABH Mice with a Combination of Protein Prenyl Transferase Inhibitors Attenuates the Severity of EAE.

(A) EAE was induced following inoculation of spinal cord homogenate in complete Freund's adjuvant at day 0 and day 7. Protein prenyl transferase inhibitors FTI-276 (free acid of FTI-277) and GGTI-297 (free acid of GGTI-298) were given daily and clinical signs of disease monitored. Data is presented as mean±SEM of clinical scores.

(B) Following remission of disease, control animals (previous clinical score of 4) and those that had received combination protein prenyl transferase therapy (previous clinical score of 0) were re-innoculated with spinal cord homogenate at day 68. Animals were monitored for clinical signs of disease daily. Data is presented as mean±SEM of clinical scores.

FIG. 4: Treatment with FTI-276 and GGTI-297 Prevents Infiltration of Leukocytes into the CNS of Biozzi ABH Mice Following Induction of EAE.

(A) EAE was induced as described in the text. Vehicle or protein prenyl transferase inhibitors FTI-276 and GGTI-297 were given daily until the animals were sacrificed at day 19. Brain and spinal cord were immersion fixed, sectioned and stained with haematoxylin and eosin for histological assessment and leukocyte marker antibodies. (a) Spinal cord, (c) cerebrum, (e) cerebral vessel and (g) cerebellum from Biozzi ABH mouse with active EAE showing marked perivascular leukocyte cuffing. (b) Spinal cord, (d) cerebrum, (f) cerebral vessel and (h) cerebellum from mouse treated with combination protein prenyl transferase inhibitors with absence of perivascular leukocyte cuffing or leukocyte infiltration into the parenchyma. a, b, g and h original magnification ×100. c and d ×200. e and f ×400

(B) Spinal cord from mouse with active EAE (panels i and k) and treated with combination protein prenyltransferase inhibitors (panels j and l). Panels i and j are stained with the T-lymphocyte marker CD3. Panels k and l are stained with a rat anti-mouse macrophage marker MOMA-2. Original magnification ×200

Arrows shows active lesions (perivascular cuffing) in animals showing active EAE. Triangles show similar vessels in section from mice treated with protein prenyl transferase inhibitors.

TABLE 1

| Clinical grade | Score | Untreated | Vehicle | PTI |
|---|---|---|---|---|
| Complete Hindlimb Paralysis | (4) | 11 | 10 | 2 |
| Partial Hindlimb Paralysis | (3) | 2 | 3 | 1 |
| Impaired Righting Reflex | (2) | — | — | 1 |
| Limp Tail | (1) | — | — | 2 |
| Normal | (0) | 2 | 3 | 7 |

| | | | |
|---|---|---|---|
| No. EAE/Total | 13/15 (87%) | 13/16 (81%) | 6/13* (46%) |
| Mean Group Score ± SEM | 3.2 ± 0.4 | 3.0 ± 0.4 | 1.3 ± 0.5**^{#} |
| Mean EAE Score ± SEM | 3.7 ± 0.1 | 3.7 ± 0.2 | 2.4 ± 0.6* |
| Mean Day of Onset ± SD | 15.1 ± 1.2 | 15.0 ± 1.5 | 15.3 ± 0.8 |

*P < 0.05, **P < 0.01 compared with untreated control
^{#}P < 0.05 compared with vehicle control

1. A protein prenyl transferase inhibitor for use in the treatment of neuroinflammatory disease.

2. The use of a protein prenyl transferase inhibitor for the preparation of medication for the treatment of neuroinflammatory disease.

3. Use according to claim 2, using a combination of a farnesyl transferase inhibitor and a geranylgeranyl transferase inhibitor.

4. Use according to claim 2 or 3, using FTI-277 and/or GGTI-298.

5. Use according to claim 2, 3, or 4, in which the disease is MS.

6. Use according to claim 2, 3, or 4, in which the disease is uveitis, alzheimers or neuroAIDS.

7. A method of alleviating neuroinflammatory disease which comprises administering to a patient in need thereof an effective amount of a protein prenyl transferase inhibitor.

8. A pharmaceutical composition comprising the combination of a farnesyl transferase inhibitor and a geranylgeranyl transferase inhibitor.

9. A pharmaceutical composition according to claim 8, in unit dosage form.

10. A composition according to claim 8 or 9, for parenteral administration.

11. A composition comprising a farnesyl transferase inhibitor and/or a geranylgeranyl transferase inhibitor to provide a daily dose of up to 25 mg/kg (e.g. from 10 to 25 mg) of the inhibitor or of each inhibitor used.
